(19) United States
(12) Patent Application Publication
(10) Pub. No.: US 2026/0027270 A1
(43) Pub. Date: Jan. 29, 2026

(54) IN-LINE GAS ANALYZER FOR PLEURAL DRAINAGE SYSTEM

(71) Applicant: Constantine Michael Poulos, Wilton, CT (US)

(72) Inventor: Constantine Michael Poulos, Wilton, CT (US)

(21) Appl. No.: 19/280,921

(22) Filed: Jul. 25, 2025

Related U.S. Application Data
(60) Provisional application No. 63/675,442, filed on Jul. 25, 2024.
(60) Provisional application No. 63/741,707, filed on Jan. 3, 2025.

(51) Int. Cl. A61M 1/04; A61M 1/00

(52) U.S. Cl. CPC A61M 1/04; A61M 1/73; A61M 2202/0225; A61M 2202/0492; A61M 2205/15; A61M 2205/3327; A61M 2205/35; A61M 2205/502

(57) ABSTRACT

The present disclosure comprises a novel in-line digital device for use with conventional pleural drainage systems. The device is configured to provide real-time analysis of one or more pleural gases or environmental factors, utilizing sensors positioned within an in-line housing to quantify parameters such as carbon dioxide concentration, temperature, and humidity. These metrics are used to support clinical decision-making regarding the timing of chest tube removal. The device is designed to be universally compatible with existing analog pleural drainage systems.

### RELATED APPLICATIONS

The present application claims the benefit of priority of U.S. Provisional Application 63/675,442, filed Jul. 25, 2024, and U.S. Provisional Application 63/741,707, filed Jan. 3, 2025, and the entire contents of the foregoing applications are incorporated herein by reference.

### FIELD

The present disclosure relates to pleural drainage systems commonly used in the medical field following cardiothoracic surgery or in the treatment of various pulmonary diseases. More particularly, the present disclosure relates to an in-line gas analyzer for monitoring and analyzing a pleural drainage system.

### BACKGROUND

Chest tube drainage systems are commonly used following thoracic surgery procedures to allow for drainage of the pleural space. Almost all patients undergoing thoracic surgical procedures, for example a lung resection, will have at least one chest tube placed at the time of surgery to allow fluid and air from the remaining lung to be evacuated from the pleural space to enable lung re-expansion. Other instances in which chest tubes are used for similar indications include drainage of spontaneous pneumothoraces, pleural space infections, or hemothoraces occurring after a traumatic injury.

Conventional chest tube drainage systems consist of two major components—(1) a chest tube and (2) a pleural drainage cannister. The chest tube is a plastic drain which is inserted into the pleural space of the patient between the lung and inner chest wall. The pleural drainage cannister is a commercially available device which usually consists of three separate chambers: one which allows the collection of drained fluid, a ‘water-seal’ chamber which acts as a one-way valve to allow expired air to exit the system, and a pressure regulator to which outside suction can be attached to promote drainage of the pleural space. These systems are widely utilized and ubiquitous in the thoracic surgical field.

Regardless of the indication of placement, eventually the chest tube will require removal. The timing of removal depends on a number of factors but chiefly the amount of fluid and air evacuated from the pleural space. The most common reason for prolonged chest drainage is an ‘airleak’ in which inhaled air continues to leak from the remaining lung due to parenchymal injury. Airleaks occur in more than 50% of patients after lung resection and are a major source of morbidity and prolonged hospitalization. As such, determining the earliest but safest time at which the chest tube can be removed is an important daily decision for a thoracic surgical team. If a chest tube system is removed prematurely, fluid or air may accumulate in the chest and can rapidly progress to life-threatening complications such as tension pneumothorax and subsequent cardiopulmonary collapse. If a chest tube system is left in place despite being unnecessary, hospitalization will be inappropriately prolonged leading to higher healthcare costs and the potential for nosocomial complications like infection or deep vein thrombosis.

In most instances of current practice, the decision to remove a chest tube system requires daily assessment by a healthcare practitioner and the integration of multiple patient factors. The most important factor in this decision-making is the visual inspection of the waterseal chamber of the pleural drainage cannister. Through a variety of provocative maneuvers, including deep breathing, coughing, or forced exhalation, the clinician will attempt to provoke the expiration of air from patient's pleural space and visualize its escape through the waterseal chamber. The detection of airleak in this manner is inherently qualitative and non-scientific as it relies not only on the clinician's clinical skill but a number of variable patient factors including their respiratory effort, pain level, and ability to follow explicit directions.

Accordingly, a need exists in the field for a cost-effective, universal device that can be used with existing pleural drainage systems to provide accurate, objective digital data to guide decision making for thoracic post-operative recovery.

### SUMMARY

The present disclosure provides a novel digital device configured to attach in-line to current ‘chest tube’ and pleural drainage systems. Specifically, the proposed design is positioned in-line with conventional pleural drainage systems between the chest tube component and the pleural drainage cannister. The digital device is equipped with at least one or more gas sensors to analyze expired air composition and guide decision-making based on a variety of compiled quantities. Entrained air from the pleural drainage system is exposed to device sensors embedded on the luminal interior of the device which is continuity with the patient's pleural space. The data from these sensors is then compiled for direct interpretation by the clinician or for use in an algorithm to aid in clinical decision-making to guide chest tube removal.

In one aspect, the disclosure provides an in-line gas analyzer, that includes: an inflow port;
an outflow port; a housing unit having a first end and a second end, wherein the first end is connected to the inflow port, the second end is connected to the outflow port, and the housing unit comprises a gas chamber and a fluid chamber separated by a divider; and one or more sensors operably connected to the gas chamber.

In some embodiments, the housing unit comprises a fluid impermeable valve in communication with the inflow port, the fluid chamber, and the gas chamber.

In some embodiments, the fluid impermeable valve is positioned between the inflow port and the gas chamber and the fluid chamber.

In some embodiments, the fluid impermeable valve is configured to separate a fluid from one or more gases passed from the inflow port into the central housing unit so that fluid is transported into the fluid chamber and the one or more gases are transported into the gas chamber.

In some embodiments, the fluid impermeable valve is positioned on an exterior surface of the central housing unit.

In some embodiments, the fluid impermeable valve is positioned on an interior surface of the central housing unit.

In some embodiments, the one or more sensors are located on an external surface of the central housing unit, optionally within an auxiliary housing unit.

In some embodiments, the one or more sensors are selected from the group consisting of a carbon dioxide sensor, an oxygen sensor, a nitrogen sensor, an argon sensor, a helium sensor, a neon sensor, a krypton sensor, a xenon sensor, a pressure sensor, a humidity sensor, a temperature sensor, and combinations thereof.

In some embodiments, the in-line gas analyzer further includes an onboard power supply or an onboard port to connect an external power supply.

In some embodiments, the in-line gas analyzer further includes one or more processors, a memory, one or more network interfaces, one or more display interfaces interconnected by a system bus.

In some embodiments, the one or more processors are configured to analyze one or more measurements received from the one or more sensors.

In some embodiments, the one or more network interfaces comprise mechanical, electrical, and signaling circuitry for communicating data over physical links.

In some embodiments, the one or more sensors include a carbon dioxide sensor, an oxygen sensor, a temperature sensor, or combinations thereof.

In some embodiments, the central housing unit includes a drain valve in fluid communication with the fluid chamber, wherein the drain valve includes an interface that allows fluid to be removed from the fluid, optionally by a syringe.

In some embodiments, the auxiliary housing unit includes a display configured to provide readout from the one or more sensors.

In one aspect, the disclosure provides a method of monitoring a pleural drainage system, which includes: using the above in-line gas analyzer to collect data from a patient; analyzing the collected data to determine if one or more data threshold values are exceeded; and maintaining the pleural drainage system in the patient if the one or more data threshold values are exceeded.

In some embodiments, the collected data comprises levels of carbon dioxide.

In some embodiments, the threshold values for carbon dioxide levels are greater than the values selected from the group consisting of about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm, and about 4500 ppm.

In some embodiments, the threshold value is about 4500 ppm.

In one aspect, the disclosure provides an inhaler for monitoring air leaks in a pleural drainage system of a patient, comprising: one or more tracer gases selected from the group consisting of an argon gas, a helium gas, a neon gas, a krypton gas, and a xenon gas, wherein the inhaler is configured to deliver the one or more tracer gases to the patient so that detection of the one or more tracer gases by the in-line gas analyzer of claim 1 indicates the presence of an air leak.

### BRIEF DESCRIPTION OF DRAWINGS

Some exemplary embodiments of the present disclosure are illustrated as an example and are not limited by figures of the accompanying drawing, in which like references may indicate similar elements and in which:

FIG. 1 depicts a conventional three-chamber pleural drainage system according to the prior art that is commonly placed following thoracic surgical procedures.

FIG. 2 depicts the perspective view of an exemplary in-line digital pleural gas analyzer according to the disclosure.

FIG. 3 depicts the in-line pleural gas analyzer central housing unit in cross-section, illustrating two separate chambers for fluid diversion and gas sampling using a one-way valve.

FIG. 4 depicts a sample of a sensor sampling event, data acquisition, and relay using a variety of gas sensors for varying gas characteristics.

FIG. 5 depicts an ex-vivo prototype example illustrating a simulated airleak and equilibrium.

### DETAILED DESCRIPTION

The present disclosure provides new in-line monitoring devices for use with conventional pleural drainage systems used in monitoring and analyzing exhaled gases from a patient. In the following description, for purposes of explanation, numerous specific details are set forth in order to provide a thorough understanding of the present disclosure. It will be evident, however, to one skilled in the art that the present disclosure can be practiced without these specific details. The present disclosure is to be considered as an exemplification of the disclosure and is not intended to limit the disclosure to these specific embodiments illustrated in the figures and description below.

The present disclosure is based, at least in part, on the discovery that an in-line entrained air gas analyzer may be used as a patient monitoring system to greatly increase post-operative outcomes for thoracic surgery patients by enabling quantitative expired air data to be analyzed and used to determine the earliest appropriate time to remove a chest tube system. Similar to the measurement of end-tidal carbon dioxide indicators used for endotracheal intubation, this device leverages the fact that exhaled gas has a significantly different composition than that of room air. Namely, the carbon-dioxide level of exhaled gas has >40,000 ppm while room air can range between 500-2000 ppm depending on local conditions. In addition, a device according to the present disclosure has the significant advantage of being positioned in a pleural drainage system in a manner (e.g., in-line in a chest tube) that is in continuity with the pleural space and therefore also able to measure other gas parameters like intrapleural pressure, temperature, humidity, and gas composition (e.g., levels of nitrogen, oxygen, carbon dioxide, argon, other trace gases, water vapor, and the like). A device in continuity (e.g., in-line in a chest tube) with the pleural space will allow for the acquisition of both continuous and on-demand measurements of these parameters which can then be compiled and analyzed.

### Overview

The techniques herein eliminate the subjective variability of current practices relating to existing monitoring pleural drainage systems while being compatible with existing ubiquitous analog pleural drainage systems. Other commercially available alternatives such as the Centese Thoraguard, ATMOS S201 Thorax, and Medela Thopaz+ have attempted to address this shortcoming by providing a digital pleural drainage cannister which replaces the analog pleural drainage cannisters entirely. Disadvantageously, these devices rely on pressure and volume readings using a titratable on-board suction device which limits the device's portability. Disadvantageously, these devices cannot be utilized with existing analog pleural drainage systems.

Other prior art attempts to solve the problem of monitoring and analyzing existing analog pleural drainage systems have used gas sensors to analyze the composition of gas, namely the carbon-dioxide and oxygen levels (see e.g., U.S. Pat. No. 9,545,462) in air evacuated from the pleural space. While this approach is compatible with existing analog pleural drainage canisters, it is limited in its application because it is not directly exposed to luminal expired gas and requires gas to exit the entire system (i.e., pass through the waterseal chamber of the pleural drainage canister) until it can be analyzed. This may limit its detection of small, subclinical leaks which is a similar limitation of current visual inspection methods.

The terminology herein is for the purpose of describing particular embodiments only and is not intended to be limiting of the disclosure. As used herein, the term “and/or” includes any and all combinations of one or more of the associated listed items. As used herein, the singular forms, “a”, “an”, and “the” are intended to include the plural forms as well as the singular forms, unless the context indicated otherwise. It will be further understood that the terms “comprises” and/or “compromising”, when used in this specification, specifically the presence of stated features, operations, elements, and/or components but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this disclosure belongs. It will be further understood that terms, such as those defined commonly in dictionaries should be interpreted as having a meaning that is consistent with their meaning in the context of the relevant art and the present disclosure and will not be interpreted in an idealized or overly formal sense unless expressly so defined herein.

In describing the disclosure, it will be understood that a number of techniques and steps are disclosed. Each of these has individual benefit and each can also be used in conjunction with one or more, or in some cases all, of other disclosed techniques. Accordingly, for the sake of clarity, this description will refrain from repeating every possible combination of the individual steps in an unnecessary fashion. Nevertheless, the specification and claims should be read with an understanding that such combinations are entirely within the scope of the disclosure and claims.

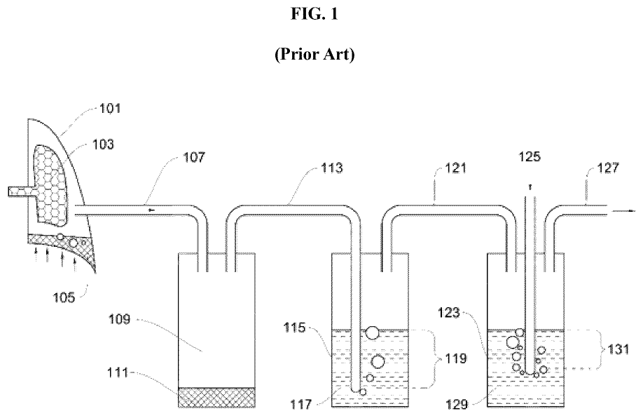

The present disclosure will now be described by referencing the appended figures representing the preferred embodiment. FIG. 1 depicts a conventional analog three-chamber pleural drainage system currently used by most thoracic surgeons and pulmonologists following procedures involving the lung and chest. This system typically consists of a chest tube 107 placed following a procedure in the chest 101. This plastic tubing drains air from the lung 103 and pleural fluid 105. The tubing is connected to the first of three chambers, chamber 109, into which expelled fluid is collected in drainage cannister 111. Expelled gas continues through tubing 113 to second waterseal chamber 115. Here, inlet tubing 113 is submerged underwater 117 which acts as a one-way valve allowing only forward passage of gas through the system. Expelled gas through this underwater seal produces visual bubbles 119 which can be observed by an observing clinician. This expelled gas then travels through tubing 121 into chamber 123 which acts as a suction regulator by use of water level 129 and room air inlet 125. External suction is applied to the system through terminal suction outlet 127. In some embodiments, our proposed device would be spliced in-line between 107 and 109 as such to be in continuity with the above system and allow analysis of intraluminal air.

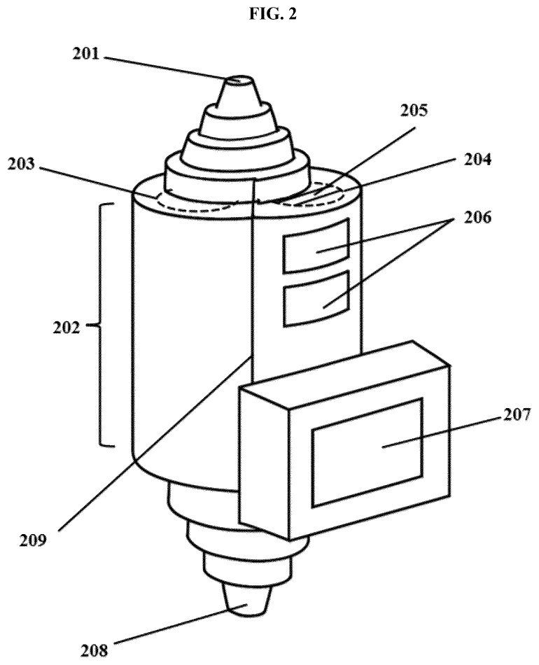

FIG. 2 depicts an exemplary embodiment of the present disclosure. In an exemplary embodiment, the device is spliced in-line between the chest tube and pleural drainage canister using two or more tubing adapters, e.g., inflow port 201 and outflow port 208, respectively, which are shown as Christmas-tree adapters in the exemplary embodiment shown in FIG. 2. It is contemplated within the scope of the disclosure that other types of tubing adapters may also be used, for example, threaded adapters, barbed adapters, push-to-connect adapters, flare fittings/adaptors, compression fittings, male and female adapters, reducing adapters, and the like. The exemplary Christmas tree adapters on either end enable the device to connect to a variety of tube sizes typically used in current practice, which usually range in measure from about 14 French to about 38 French. Entrained air and fluid from the patient's pleural space drain into the central housing unit 202. This central housing unit is bifurcated into by inner chamber divider 209 into two separate chambers—fluid chamber 203 and gas chamber 204. Using a one-way, fluid-impermeable valve 205 air is preferentially shunted to the gas chamber 204 while pleural fluid passes through the other fluid lumen 203. Entrained air is then exposed to one or more sensors 206 embedded in the wall of the central housing unit to allow for data acquisition. In some embodiments, these may be sensors for measuring gas concentration (e.g., levels of nitrogen, oxygen, carbon dioxide, argon, other trace gases, water vapor, and the like) as well as other gas parameters including pressure, temperature, and humidity. Fluid chamber 203 may include a drain valve in fluid communication with the fluid chamber and configured so the drain valve may allow the fluid chamber to be drained. For example, the drain valve may include an adaptor configured to interface with a syringe or pump either directly, or indirectly (e.g. via tubing). Fluid chamber 203 may also include a vent valve (e.g. a one-way vent valve that allows air intake into the fluid chamber) that functions in combination with the drain valve to allow fluid drainage without creating a vacuum in the fluid chamber.

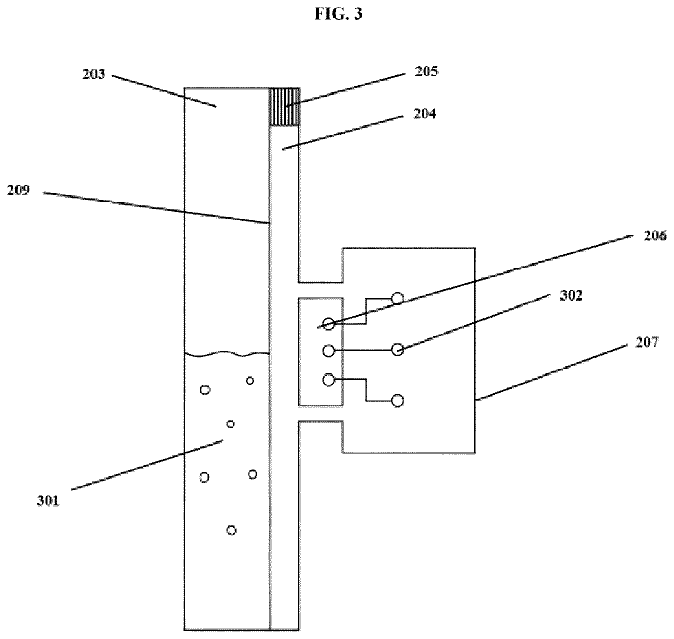

FIG. 3 depicts the central housing unit 202 in cross-section. Herein, the chamber is divided by chamber divider 209 into two separate lumens: the fluid chamber 203 and the gas chamber 204. Gas is preferentially diverted into the gas chamber by using of a one-way, fluid impermeable valve 205. In some embodiments, this valve is a hydrophobic membrane, mechanical valves including duckbill, umbrella, gate, ball, butterfly, or diaphragm designs, or orientation specific valve which allows the chamber to remain free from any evacuated pleural fluid 301. Embedded sensors 206 allow for analysis of gas sampling from the gas chamber for further compilation and processing by electronic component 302 which is contained in the auxiliary housing unit 207. The auxiliary housing unit contains all necessary electrical components required for power, data and acquisition, processing, and display.

For example, the housing unit may include a simplified computing system comprising one or more processors, a memory, one or more network interfaces, one or more display interfaces interconnected by a system bus. The memory may include a plurality of storage locations that are addressable by the one or more processor and the network interfaces for storing software programs and data structures associated with use of the one or more sensors described herein. The processor may comprise hardware elements or hardware logic adapted to execute the software programs and manipulate the data generated by the one or more sensors. An operating system, portions of which are typically resident in the memory and executed by the processor, serves to handle I/O data streams generated by the one or more sensors and invokes operations in support of software processes and/or services executing on the device to include one or more functional processes. For example, functional processes executed by the one or more processors can analyze data generated by the one or more sensors, generate current values for one or more factors (e.g., temperature, pressure, gas levels (e.g., ppm), and the like) and determine whether or not the identified current values are lower than, equal to, or higher than specified threshold values, thereby performing various functions corresponding to the in-line gas analyzer's purpose and general configuration.

It will be apparent to those skilled in the art that other processor and memory types, including various computer-readable media, may be used to store and execute program instructions pertaining to the techniques herein. Also, while the disclosure illustrates various processes, it is expressly contemplated that various processes may be embodied as modules configured to operate in accordance with the techniques herein (e.g., according to the functionality of a similar process). Further, while the processes have been shown separately, those skilled in the art will appreciate that processes may be routines or modules within other processes.

It is contemplated that the disclosed in-line gas analyzer may interface (e.g., via a network interface) with any number of client devices (e.g., computers, tablets, phones, etc.), one or more servers, and one or more databases, where the devices may be in communication with one another via any number of networks. The one or more networks may include any number of specialized networking devices such as routers, switches, access points, and the like that interconnected via wired and/or wireless connections. For example, in-line gas analyzer and/or the intermediary devices in the above network(s) may communicate wirelessly via links based on WiFi, cellular, infrared, radio, near-field communication, satellite, or the like. Other such connections may use hardwired links, e.g., Ethernet, fiber optic, etc. The nodes/devices typically communicate over the network by exchanging discrete frames or packets of data (packets 140) according to predefined protocols, such as the Transmission Control Protocol/Internet Protocol (TCP/IP) other suitable data structures, protocols, and/or signals. It will be appreciated by the skilled artisan that a protocol consists of a set of rules defining how the nodes interact with each other.

Client devices may include any number of user devices or end point devices configured to interface with the techniques herein. For example, client devices may include, but are not limited to, desktop computers, laptop computers, tablet devices, smart phones, wearable devices (e.g., heads up devices, smart watches, etc.), set-top devices, smart televisions, Internet of Things (IoT) devices, autonomous devices, collaboration endpoints, or any other form of computing device capable of participating with other devices via network(s).

The in-line gas analyzer may include a display interface that may include the mechanical, electrical, and signaling circuitry for displaying and/or capturing video signals (e.g., current values detected by the one or more sensors). In various embodiments, the device is operatively powered by a range of electrical sources. In some embodiments, the power supply comprises a 5-volt direct current (DC) input, such as that provided via a USB Type-C connector, including but not limited to commercially available wall adapters or power sources commonly used with single-board computers. In other embodiments, the device may include an integrated power supply comprising one or more lithium-ion rechargeable batteries. Alternatively, the device may be powered by standard non-rechargeable or rechargeable dry-cell batteries, including but not limited to AA or AAA form factors. The power supply may be housed within the device body or externally connected via appropriate terminals or connectors.

In various embodiments, the device is configured to acquire, process, and transmit data using one or more computational and communication systems. In some embodiments, data processing is performed locally using an onboard microprocessor or microcontroller, which may include but is not limited to single-board computers, application-specific integrated circuits, or system-on-chip platforms. In alternative embodiments, the device transmits raw or partially processed data to an external computing system for analysis. Such transmission may occur via wireless communication protocols including, but not limited to, Bluetooth, Wi-Fi, Zigbee, or other radio frequency (RF)-based schemes. In certain embodiments, the device further includes an integrated visual display, which may comprise an LCD, OLED, or e-ink screen, configured to present real-time or summary data and to facilitate user interaction. The display may additionally serve as a user interface for configuration, control, or feedback purposes.

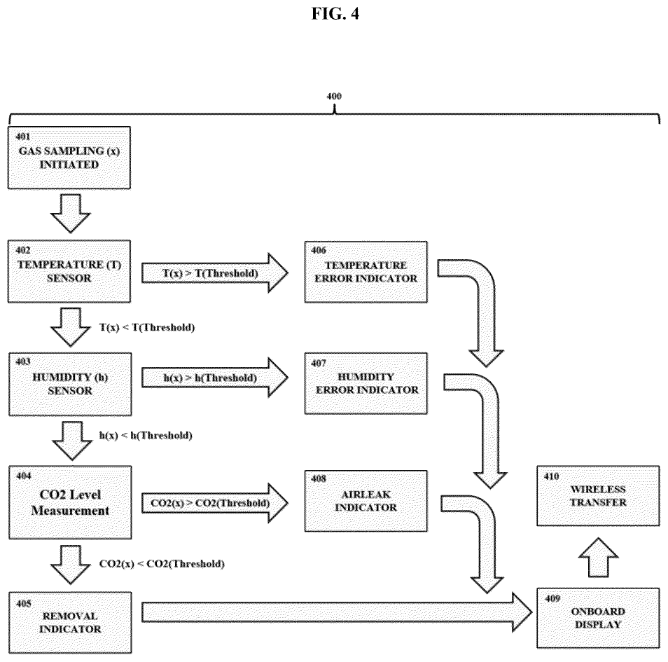

FIG. 4 is a flow chart detailing the method 400 of data acquisition and reporting. In block 401, a gas sampling event is initiated. In some embodiments, this may be initiated by the clinician using an on-board button input. In other embodiments, the device may be calibrated to conduct measurements continuously or at pre-determined intervals depending on the clinical scenario. In block 402, a temperature measurement is conducted using the embedded temperature sensor to ensure nominal temperature readings. If this temperature reading is above the predetermined temperature threshold indicating a potential device failure, a temperature error indicator 406 will be activated. In some embodiments this will be displayed on onboard display as shown in block 409 and in some other embodiments, this will be further relayed to another device wirelessly as shown in block 410. If the temperature measurement is within normal limits, at block 403, a humidity measurement will be initiated by embedded humidity sensor. If this humidity reading is above the predetermined humidity threshold indicating potential contamination, a humidity error indicator 407 will be activated. In some embodiments this will be displayed on onboard display as shown in block 409 and in some other embodiments, this will be further relayed to another device wirelessly as shown in block 410. Lastly, a carbon dioxide measurement 404 will be taken. If the carbon dioxide measurement is above the predetermined threshold indicating an ongoing airleak, an airleak indicator 408 will be activated. In some embodiments this will be displayed on onboard display as shown in block 409 and in some other embodiments, this will be further relayed to another device wirelessly as shown in block 410. If there is a low carbon-dioxide level that does not meet the predetermined threshold, a removal indicator 405 will be activated. In some embodiments this will be displayed on onboard display as shown in block 409 and in some other embodiments, this will be further relayed to another device wirelessly as shown in block 410.

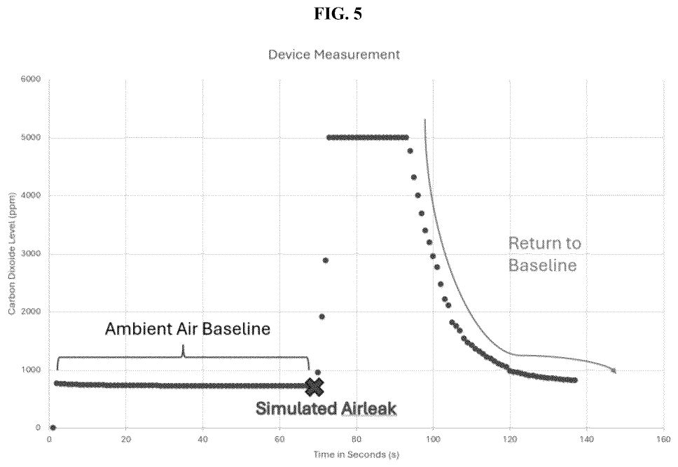

FIG. 5 displays a simulated, ex-vivo example of this device's operation.

In operation, gas collected from the pleural space is continuously or intermittently sampled and analyzed. If the measured carbon dioxide concentration exceeds a predetermined threshold (e.g., about >3000, about >3100, about >3200, about >3300, about >3400, about >3500, about >3600, about >3700, about >3800, about >3900, about >4000, about >4100, about >4200, about >4300, about >4400, or about >4500 ppm), this is indicative of ongoing airleak, and, as such, the clinician should opt to continue chest tube drainage until resolution. One of skill in the art will appreciate that the specific threshold chosen to make this decision may be varied within the disclosed ranges or values based on environmental conditions that may affect CO₂ levels such as air pressure, temperature, elevation, and the like.

Conversely, if carbon dioxide concentrations are below the threshold, this may suggest resolution of the air leak, allowing for consideration of tube removal by the managing clinician. Normal CO₂ levels for room air can range from 400-1500 ppm depending on ventilation while exhaled air typically ranges from 40,000-50,000 ppm.

CO₂ levels in indoor environments vary based on several factors, most notably ventilation, occupancy, and air exchange rate. Generally, room air CO₂ concentrations range from about 400-1,500 ppm. At the lower end, freshly ventilated spaces typically exhibit levels around 400-750 ppm, while more poorly ventilated areas may rise into the 800-1,200 ppm range. Spaces with very limited ventilation or high occupancy may even exceed 1,500 ppm. More granularly, nested sub-ranges include 400-600 ppm, 600-800 ppm, 800-1,000 ppm, and 1,000-1,500 ppm, each corresponding to increasing degrees of ventilation inadequacy and occupant density. Specific values at 10 ppm intervals often referenced for indoor air quality monitoring include: about 400, 410, 420, 430, 440, 450, 460, 470, 480, 490, and 500 ppm for well-ventilated spaces; and about 510, 520, 530, 540, 550, 560, 570, 580, 590, 600 ppm in slightly less ideal conditions. As CO₂ rises beyond this, moderate accumulation is represented by about 610-800 ppm, with thresholds like 700, 750, 800, 850, 900, 950, 1,000 ppm providing important benchmarks. High levels, such as about 1,100, 1,200, 1,300, 1,400, 1,500 ppm, indicate urgent ventilation needs.

Exhaled human breath contains significantly elevated CO₂ concentrations compared to ambient air due to respiratory metabolism. Typical ranges for exhaled CO₂ lie between about 40,000-50,000 ppm, or 4-5% by volume. Within this broader range, individual variations and measurement precision allow for breakdowns such as 40,000-42,000 ppm, 42,000-45,000 ppm, and 45,000-50,000 ppm. Specific values commonly observed include about 40,000, 40,100, 40,200, 40,300, 40,400, 40,500, 40,600, 40,700, 40,800, 40,900, 41,000, continuing at regular intervals such as 41,500, 42,000, 42,500, 43,000, and so on up to 50,000 ppm. The variability is influenced by factors like exertion level, respiratory health, and measurement technique. For example, a resting adult might exhale CO₂ at about 40,000-45,000 ppm, while levels during physical activity or speaking may approach 48,000-50,000 ppm.

To ensure the reliability of CO₂ measurements, additional environmental sensors may be incorporated to detect temperature and humidity. Abnormal readings from these sensors may indicate contamination of the gas lumen by intrathoracic fluid (e.g., blood, pleural effusion), which may compromise the accuracy of gas analysis. In such cases, or as part of standard verification, the downstream waterseal chamber 115 of the traditional pleural drainage canister can be inspected to verify the presence or absence of airleak. Additional sensors that detect oxygen, nitrogen sensors, argon and other trace gases typically present in exhaled air may also be included. It is specifically contemplated within the disclosure that specific combinations of sensors may be included on central housing unit 202.

In this specific example, the intraluminal sensor measured carbon-dioxide at a fixed interval of 1 Hertz. The device is calibrated to ambient air. A simulated airleak of expired air was induced into the system resulting in a rapid rise in intraluminal carbon-dioxide levels, as measured in parts-per-million. The sensor reaches its maximum detection level of 5000 and plateaus for approximately 30 seconds. After this latent period, the system returns to equilibrium.

The techniques herein also provide that central housing unit 202 may include sensors for inert gases that either are, or are not, normally present in exhaled air that could be used as tracer gases to increase the sensitivity of detection of an airleak such as, for example, argon, helium, neon, krypton, xenon, or the like. Such gases can be provided to the patient by way of an inhaler that would allow inspiration of a small amount of tracer gas, the presence or absence of which could be immediately assessed by the sensor of the present disclosure.

What is claimed is:

1. An in-line gas analyzer, comprising:
an inflow port;
an outflow port;
a housing unit having a first end and a second end, wherein the first end is connected to the inflow port, the second end is connected to the outflow port, and the housing unit comprises a gas chamber and a fluid chamber separated by a divider; and
one or more sensors operably connected to the gas chamber.

2. The in-line gas analyzer of claim 1, wherein the housing unit comprises a fluid impermeable valve in communication with the inflow port, the fluid chamber, and the gas chamber.

3. The in-line gas analyzer of claim 2, wherein the fluid impermeable valve is positioned between the inflow port and the gas chamber and the fluid chamber.

4. The in-line gas analyzer of claim 2, wherein the fluid impermeable valve is configured to separate a fluid from one or more gases passed from the inflow port into the central housing unit so that fluid is transported into the fluid chamber and the one or more gases are transported into the gas chamber.

5. The in-line gas analyzer of claim 2, wherein the fluid impermeable valve is positioned on an exterior surface of the central housing unit.

6. The in-line gas analyzer of claim 2, wherein the fluid impermeable valve is positioned on an interior surface of the central housing unit.

7. The in-line gas analyzer of claim 1, wherein the one or more sensors are located on an external surface of the central housing unit, optionally within an auxiliary housing unit.

8. The in-line gas analyzer of claim 1, wherein the one or more sensors are selected from the group consisting of a carbon dioxide sensor, an oxygen sensor, a nitrogen sensor, an argon sensor, a helium sensor, a neon sensor, a krypton sensor, a xenon sensor, a pressure sensor, a humidity sensor, a temperature sensor, and combinations thereof.

9. The in-line gas analyzer of claim 1, further comprising an onboard power supply or an onboard port to connect an external power supply.

10. The in-line gas analyzer of claim 1, further comprising one or more processors, a memory, one or more network interfaces, one or more display interfaces interconnected by a system bus.

11. The in-line gas analyzer of claim 10, wherein the one or more processors are configured to analyze one or more measurements received from the one or more sensors.

12. The in-line gas analyzer of claim 10, wherein the one or more network interfaces comprise mechanical, electrical, and signaling circuitry for communicating data over physical links.

13. The in-line gas analyzer of claim 1, wherein the one or more sensors include a carbon dioxide sensor, an oxygen sensor, a temperature sensor, or combinations thereof.

14. The in-line gas analyzer of claim 1, wherein the central housing unit includes a drain valve in fluid communication with the fluid chamber, wherein the drain valve includes an interface that allows fluid to be removed from the fluid, optionally by a syringe.

15. The in-line gas analyzer of claim 7, wherein the auxiliary housing unit includes a display configured to provide readout from the one or more sensors.

16. A method of monitoring a pleural drainage system, comprising:
using the in-line gas analyzer of claim 1 to collect data from a patient;
analyzing the collected data to determine if one or more data threshold values are exceeded; and
maintaining the pleural drainage system in the patient if the one or more data threshold values are exceeded.

17. The method of claim 16, wherein the collected data comprises levels of carbon dioxide.

18. The method of claim 17, wherein the threshold values for carbon dioxide levels are greater than the values selected from the group consisting of about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm, and about 4500 ppm.

19. The method of claim 18, wherein the threshold value is about 4500 ppm.

20. An inhaler for monitoring air leaks in a pleural drainage system of a patient, comprising: one or more tracer gases selected from the group consisting of an argon gas, a helium gas, a neon gas, a krypton gas, and a xenon gas, wherein the inhaler is configured to deliver the one or more tracer gases to the patient so that detection of the one or more tracer gases by the in-line gas analyzer of claim 1 indicates the presence of an air leak.
